(19) United States
(12) Patent Application Publication
(10) Pub. No.: US 2015/0342222 A1
(43) Pub. Date: Dec. 3, 2015

(54) NOVEL USE OF CANTHAXANTHIN AND 25-HYDROXY VITAMIN D3

(71) Applicant: DSM IP ASSETS B.V., Te Heerlen (NL)

(72) Inventors: Rual LOPEZ ULIBARRI, Kaiseraugst (CH); Isaac BITTAR, Kaiseraugst (CH); Jose-Otavio B. SORBARA, Kaiseraugst (CH)

(21) Appl. No.: 14/655,115

(22) PCT Filed: Dec. 12, 2013
(86) PCT No.: PCT/IB2013/060844

§ 371 Date: Jun. 24, 2015

(30) Foreign Application Priority Data
Dec. 27, 2012 (EP) 12199398.4
May 31, 2013 (EP) 13170040.3

(51) Int. Cl. A23K 1/16; A61K 31/047; A23K 1/18; A61K 31/122

(52) U.S. Cl. CPC A23K 1/1603; A61K 31/122; A61K 31/047; A23K 1/1826; A23K 1/1646

(57) ABSTRACT

The present invention relates to the use of canthaxanthin and at least one vitamin D metabolite, preferably 25-hydroxy vitamin D3 (25-OH D3), for improving broiler performance. More particularly the invention relates to the use of canthaxanthin and 25-hydroxy vitamin D3 in the manufacture of a feed composition for improving weight gain and carcass ratio.

### FIELD OF THE INVENTION

The present invention relates to the use of canthaxanthin and at least one vitamin D metabolite, preferably 25-hydroxy vitamin D3 (25-OH D3), for improving broiler performance. More particularly the invention relates to the use of canthaxanthin and 25-hydroxy vitamin D3 in the manufacture of a feed composition for improving weight gain in poultry broilers and increasing carcass ratio of poultry meet.

### BACKGROUND

In accordance with the present invention it has been found that feed intake, weight gain and feed conversion ratio can be improved in poultry broilers by administering to the animals an effective amount of Canthaxanthin and 25-OH-D3.

Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline food. This composition stabilizes 25-OH D3 against oxidation.

Simoes-Nunes et al. (US 2005/0064018) discloses adding a combination of 25-OH Vitamin D3 and Vitamin D3 to animal feed. In particular, about 10 μg/kg to about 100 μg/kg of 25-OH Vitamin D3 and about 200 IU/kg to about 4,000 IU/kg of Vitamin D3 are added to swine feed. This addition improves the pig's bone strength.

Stark et al. (U.S. Pat. No. 5,695,794) disclose adding a combination of 25-OH Vitamin D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1alpha, 25-dihydroxycholecalciferol to improve egg strength and leg strength in laying hens and older hens.

Fleshner-Barak (WO 03/007916) discloses administration of bisphosphonate compound and natural vitamin D derivative such as 1,25-dihydroxyvitamin D3 or 24,25-dihydroxyvitamin D3, or 25-OH vitamin D3.

Daifotis et al. (WO 03/086415) disclose inhibiting bone resorption by a combination of at least one bisphosphonate compound and from about 100 IU to about 60,000 IU of a no activated metabolite of vitamin D2 and/or vitamin D3.

The aforementioned documents did not teach or suggest that the use of canthaxanthin and 25-OH D3 would be surprisingly beneficial for improving feed intake, weight gain and feed conversion ratio in poultry broilers.

### DETAILED DESCRIPTION OF THE INVENTION

As used throughout the specification and claims, the following definitions apply:

“Vitamin D metabolite” means any metabolite of Vitamin D as for example 25-hydroxy vitamin D3, 1,25-dihydroxy vitamin D3 or 24,25-dihydroxy vitamin D3.

“25-OH D3” refers specifically to 25-hydroxy vitamin D3.

“Poultry” is meant to include turkeys, ducks and chickens (including but not limited to broiler chicks, layers, breeders).

Canthaxanthin and 25-OH D3 may be obtained from any source, and a composition thereof may be prepared using convenient technology.

The term carcass means the body of an animal, that has been slaughtered for food, with the head, limbs, and entrails removed. The term carcass ratio is determined on the basis of the body weight that has been slaughtered for food, with the head, limbs, and entrails removed and of the body weight of the animal before slaughtering.

In a first aspect, one or more feed compositions suitable for poultry use are provided to administer canthaxanthin and 25-OH D3 as nutrients to improve weight gain in poultry broilers.

In a second aspect, one or more feed compositions suitable for poultry use are provided to administer canthaxanthin and 25-OH D3 as nutrients to increase carcass ratio in poultry animals.

In a third aspect, a poultry feed is provided which comprises from about 10 μg/kg to about 100 μg/kg of 25-OH D3 and from about 2 to 100 ppm canthaxanthin, preferably 2 to 10 ppm.

In another aspect, a method of administering canthaxanthin and 25-OH D3 to poultry broilers is provided to improve weight gain and carcass ratio in poultry animals, e.g. broilers. In particular, the method comprises administering to the animal in need of such treatment an amount of about 2 ppm to 100 ppm of canthaxanthin, preferably 2 to 10 ppm, and about 10 μg/kg to about 100 μg/kg of 25-OH D3.

In another aspect, a premix composition for poultry feed comprising 25-hydroxy vitamin D3 and canthaxanthin is provided.

Canthaxanthin and 25-hydroxy vitamin D3 are suitably administered together with the food. The term food as used herein comprises both solid and liquid food as well as drinking fluids such as drinking water. Particularly, inventive ingredients can be added as a formulated powder to a premix containing other minerals, vitamins, amino acids and trace elements which is added to regular animal food and thorough mixing to achieve even distribution therein.

In the manufacture of poultry feed in accordance with the invention, from about 2 ppm to 100 ppm, preferably 2-10 ppm of canthaxanthin and from about 10 μ/kg to about 100 μg/kg of 25-hydroxy vitamin D3 are added to regular poultry food. Alternatively, a food premix may be prepared on the basis of regular food components by adding these active ingredients to such food components in higher concentration.

According to the present invention the 25-OH-D3 compound is available under the Trademark ROVIMIX® Hy-D® 1.25% and canthaxanthin under the Trademark CAROPHYLL®Red. A combination product of both, canthaxanthin and 25-OH-D3 is available under the Trademark MAXICHICK®.

According to the present invention it is further advantageous if the composition also contains one or more of the following ingredients: Vitamin A, Vitamin E, Biotin, copper (e.g. as CuSO₄), zinc (e.g. as ZnSO₄), cobalt (e.g. as CoSO₄), selenium (e.g. as Na₂SeO₃), iodine (e.g. as KI), manganese (e.g. as MnSO₄) and/or calcium (e.g. as CaSO₄).

The following non-limiting Examples are presented to better illustrate the invention.

### EXAMPLE 1

### Effect of Carophyll Red (Canthaxanthin) and 25-OH D3 on Feed Intake, Weight Gain and Feed Conversion Ratio of Poultry Broilers

Material & Methods

An experiment has been conducted with 240 young chicks. The trial had a completely randomized factorial 2×2 design, wherein two sources of progeny have been used:

a) chicks from mother breeders supplemented with Maxichick

b) chicks form mother breeders not supplemented with Maxichick

and wherein the trial has been designed with four treatments and 8 replicates of 15 birds.

The trial had three stages at three different ages: 35, 45 and 55 weeks.

Feeding

The diet given to the birds was a standard feed for broiler chickens with the addition of the products that were tested. The feed met all nutritional requirements in relation to the developmental stage of the birds and the recommendations in the breeders' guide. The feed was entirely vegetable-based, using corn and Soya bran (An example of a premix diet without Maxichick is given in Table 7).

TABLE 1
Treatments used in the experiment on broiler chickens.

| | | 25-OH-D3 |
|---|---|---|
| Treatments | Canthaxanthin (ppm) | (ppm) |
| Control | 0 | 0 |
| Maxichick | 6 mg/ton (6 ppm given as 60 ppm | 69 ug/ton |
| | CAROPHYLL ®Red 10%) | |

Experimental Design

The Experimental Design was Entirely Random, with Two Treatments and Six Groups of 30 Female and 3 Male Chickens.

Methodology

Weight gain, feed intake and feed conversion ratio was calculated according methods known to the person skilled in the art.

After the data was obtained, a variation analysis was carried and standard deviation was calculated. These statistical procedures were carried out with the help of the statistical program SAS.

Results

TABLE 2
Progeny performance from breeders fed maxichick (35 wks)

| | | | |
|---|---|---|---|
| Progeny | Control | Maxichick | Average |

Broiler Breeders
Feed Intake, g

| | | | |
|---|---|---|---|
| Control* | 4.927 | 4.918 | 4.923 |
| Maxichick** | 4.913 | 4.999 | 4.906 |
| Average | 4.920 | 4.909 | |

Weight gain, g

| | | | |
|---|---|---|---|
| Control* | 2.857 | 2.913 | 2.885 |
| Maxichick* | 2.864 | 2.981 | 2.923 |
| Average | 2861 | 2.947 | |

Feed Conversion, g/g

| | | | |
|---|---|---|---|
| Control* | 1.72 | 1.69 | 1.71 |
| Maxichick** | 1.72 | 1.68 | 1.70 |
| Average | 1.72^{B} | 1.69^{A} | |

*Mother breeders not supplemented with Maxichick
**Mother breeder supplemented with Maxichick

TABLE 3
Progeny carcass characteristics from breeders fed maxichick (35 wks)

| | | | |
|---|---|---|---|
| Progeny | Control | Maxichick | Average |

Broiler Breeders
Carcass, %

| | | | |
|---|---|---|---|
| Control* | 69.50 | 70.33 | 69.92 |
| Maxichick** | 70.88 | 71.59 | 71.24 |
| Average | 70.19 | 70.96 | |

Breast, %

| | | | |
|---|---|---|---|
| Control* | 30.51 | 31.13 | 30.82 |
| Maxichick** | 31.09 | 31.86 | 31.48 |
| Average | 30.08 | 31.50 | |

Legs, %

| | | | |
|---|---|---|---|
| Control* | 31.98 | 31.61 | 31.80 |
| Maxichick** | 31.99 | 31.86 | 31.93 |
| Average | 31.99 | 31.74 | |

TABLE 4
Progeny performance from breeders fred maxichick (45 wks)

| | | | |
|---|---|---|---|
| Progeny | Control | Maxichick | Average |

Broiler Breeders
Feed Intake, g

| | | | |
|---|---|---|---|
| Control* | 4.815 | 4.904 | 4.860 |
| Maxichick** | 4.899 | 4.953 | 4.926 |
| Average | 4.857 | 4.929 | |

Weight gain, g

| | | | |
|---|---|---|---|
| Control* | 2.873 | 2.947 | 2.910 |
| Maxichick** | 2.862 | 2.994 | 2.928 |
| Average | 2868 | 2.971 | |

Feed Conversion, g/g

| | | | |
|---|---|---|---|
| Control* | 1.68 | 1.66 | 1.67 |
| Maxichick** | 1.71 | 1.65 | 1.68 |
| Average | 1.70 | 1.66 | |

TABLE 5
Progeny carcass characteristics from breeders fed maxichick (45 wks)

| | | | |
|---|---|---|---|
| Progeny | Control | Maxichick | Average |

Broiler Breeders
Carcass, %

| | | | |
|---|---|---|---|
| Control* | 70.14 | 71.22 | 70.68 |
| Maxichick** | 70.64 | 72.34 | 71.21 |
| Average | 70.39 | 71.78 | |

Breast, %

| | | | |
|---|---|---|---|
| Control* | 30.44 | 31.87 | 31.16 |
| Maxichick** | 30.84 | 31.99 | 31.42 |
| Average | 30.64 | 31.93 | |

Legs, %

| | | | |
|---|---|---|---|
| Control* | 31.17 | 31.75 | 31.46 |
| Maxichick** | 31.24 | 31.92 | 31.58 |
| Average | 31.21 | 31.84 | |

TABLE 6
Progeny performance from breeders fed maxichick (62 wks)

| | | | |
|---|---|---|---|
| Progeny | Control | Maxichick | Average |

Broiler Breeders
Feed Intake, g

| | | | |
|---|---|---|---|
| Control* | 4.944 | 4.927 | 4.936 |
| Maxichick** | 4.977 | 4.981 | 4.979 |
| Average | 4.961 | 4.954 | |

Weight gain, g

| | | | |
|---|---|---|---|
| Control* | 2.830 | 2.945 | 2.888 |
| Maxichick** | 2.853 | 2.989 | 2.921 |
| Average | 2842 | 2.967 | |

Feed Conversion, g/g

| | | | |
|---|---|---|---|
| Control* | 1.75 | 1.67 | 1.71 |
| Maxichick** | 1.74 | 1.67 | 1.71 |
| Average | 1.75 | 1.67 | |

TABLE 7
Progeny carcass characteristics from breeders fed maxichick (62 wks)

| | | | |
|---|---|---|---|
| Progeny | Control | Maxichick | Average |

Broiler Breeders
Carcass, %

| | | | |
|---|---|---|---|
| Control* | 70.04 | 71.23 | 70.64 |
| Maxichick** | 70.98 | 72.23 | 71.61 |
| Average | 70.51 | 70.73 | |

Breast, %

| | | | |
|---|---|---|---|
| Control* | 30.23 | 31.44 | 30.84 |
| Maxichick** | 31.02 | 31.88 | 31.45 |
| Average | 30.63 | 31.66 | |

Legs, %

| | | | |
|---|---|---|---|
| Control* | 31.83 | 31.59 | 31.71 |
| Maxichick** | 31.74 | 31.92 | 31.83 |
| Average | 31.79 | 31.76 | |

TABLE 8
Composition of the premix added to the diet of the chickens

| Nutrient | Quantity per kilo of product | Unit |
|---|---|---|
| Folic Acid | 237.5 | mg |
| Nicotinic Acid | 8500 | mg |
| Pantothenic Acid | 3800 | mg |
| Biotin | 38 | mg |
| Copper | 12400 | mg |
| Choline | 72000 | mg |
| Sulphur | 10222 | mg |
| Iron | 12000 | mg |
| Iodine | 160 | mg |
| Manganese | 14000 | mg |
| Methionine | 118800 | mg |
| Oxytetracycline | 8000 | mg |
| Selenium | 108 | mg |
| Vitamin A | 2090000 | UI/Kg |
| Vitamin B1 | 475 | mg |
| Vitamin B12 | 3800 | mg |
| Vitamin B2 | 1900 | mg |
| Vitamin B6 | 950 | mg |
| Vitamin E | 7600 | mg |
| Vitamin K3 | 950 | mg |
| Zinc | 14000 | mg |

1. The use of canthaxanthin and at least one vitamin D metabolite for improving weight gain in poultry broilers.

2. The use of canthaxanthin and at least one vitamin D metabolite for improving carcass ratio in poultry.

3. The use according to claim 1, wherein the vitamin D metabolite is 25-hydroxy vitamin D3.

4. The use of canthaxanthin and or 25-hydroxy vitamin D3 in the manufacture of a food or veterinary composition for improving weight gain in poultry broilers and carcass ratio in poultry animals.

5. The use as in claim 1 in the manufacture of a poultry food comprising from about 10 μg/kg to about 100 μg/kg of 25-hydroxy vitamin D3 and from about 2 to 100 ppm canthaxanthin, preferably from about 2 ppm to 10 ppm.

6. A method for improving weight gain carcass ratio in poultry, which comprises administering to an animal in need of such treatment an amount of about 2 ppm to 100 ppm, preferably 2 to 10 ppm of canthaxanthin and/or about 10 μg/kg to about 100 μg/kg of at least one vitamin D metabolite.

7. The method according to claim 6, wherein the vitamin D metabolite is 25-hydroxy vitamin D3.

8. The method according to claim 6, wherein canthaxanthin and 25-hydroxy vitamin D3 are administered together.

9. A premix composition comprising 25-hydroxy vitamin D3 and canthaxanthin for use in broiler feed for improving weight gain and carcass ratio in poultry animals.
